(19) United States
(12) Patent Application Publication
(10) Pub. No.: US 2008/0206359 A1
(43) Pub. Date: Aug. 28, 2008

(54) Day and Night Composition and method for treating Pseudofolliculitis Barbae

(76) Inventor: Nikita Wilson, (US)

Correspondence Address: Nikita Wilson, 408 Wallingford Terrace, Union, NJ, 07083, US

(21) Appl. No.: 11/678,096

(22) Filed: Feb. 23, 2007

(51) Int. Cl. A61K 31/191; A61K 31/047; A61K 31/4166; A61K 31/695; A61K 31/7016; A61K 33/00; A61K 33/06; A61K 36/886; A61K 33/14; A61K 33/02; A61K 31/717; A61K 31/7004; A61K 31/566; A61K 31/19

(52) U.S. Cl. 424/666; 424/682; 424/719; 424/722; 424/744; 514/178; 514/25; 514/35; 514/385; 514/557; 514/568; 514/574; 514/63; 514/729

(57) ABSTRACT

An effective formulation for the treatment and prevention of pseudofolliculitis barbae, commonly called razor bumps, and the skin irritation associated with the condition. The treat formulation can be used as the last step in a multi-step shaving system consisting of a shaving gel, aftershave balm and the aforementioned treat. The treat can also be used as a standalone product and is formulated to treat and prevent shaving symptoms day and night.

### FIELD OF THE INVENTION

The present invention relates generally to the treatment of pseudofolliculitis barbae and in the treatment and prevention of skin irritation, ingrown hair and razor bumps. The invention is a topical balm or cream designed to eliminate razor bumps and multiple shaving symptoms that develops on the skin of some individuals after shaving while treating and preparing the skin for subsequent shavings. The compound is applied most preferably as a component of a cosmetically acceptable balm after shaving or during the days when not shaving and nights for razor symptom prevention.

### BACKGROUND OF THE INVENTION

Pseudofolliculitis barbae is a common condition of the beard area occurring most often in people with curly hair. The condition is commonly referred to as “razor bumps”. Recent studies suggest there may be two reasons why razor bumps occur. One is when highly curved hairs grow back into the skin causing inflammation and a foreign body reaction. This can cause keloidal scarring which looks like hard bumps on the shaved area.

The other theory of the occurrence of Pseudofolliculitis barbae states that “razor bumps” are a direct manifestation of inflammation caused by the skins reaction to the trauma caused by shaving. Therefore, a treatment is needed to accommodate both causes of pseudofolliculitis barbae, inflammation and other razor symptoms.

### DESCRIPTION OF THE RELATED ART

Various compounds have been developed for the treatment of pseudofolliculitis barbae. Although many of the compounds are of different composition, many of them are ineffective in resolving the issues of razor bumps as they occur. Pseudofolliculitis barbae and other shaving symptoms continue to be a problem for many people. This is why a new, more effective solution is warranted.

Some pseudofolliculitis barbae remedies involve the application of depilatories and other topical agents. These formulas either use harsh chemicals to remove hair without shaving or only attempt to prevent inflammation of the skin due to ingrown or curling hairs. The depilatory compounds are often unpleasant to use and can result in severe drying of the skin and the topical formulas do not resolve the issue of skin inflammation due to shaving trauma.

Furthermore, other chemical compounds have been designed to treat the user's skin after shaving with a standard razor to prevent pseudofolliculitis barbae; while the present treatment method is used as a day and night treat for any shaving method.

For example, U.S. Pat. Nos. 6,703,009 and 5,035,221 contains isopropyl alcohol and acetylsalicylic acid which may dry the skin and cause further irritation with extended use. U.S. Pat. No. 6,352,690 contains benzoyl peroxide derivatives and various oils which are ineffective in treating inflammation and outbreaks. Repeated use of these chemical compounds can add additional irritation to the user. Further, the above patents have limited effectiveness eliminating pseudofolliculitis barbae and instead of treating pseudofolliculitis barbae they can potentially cause more damage.

The present treatment method overcomes the problems enumerated above.

### SUMMARY OF THE INVENTION

The primary object of the present invention is to provide an absolute effective method for the treatment of pseudofolliculitis barbae and shaving symptoms. This is accomplished by treating both reasons for inflammation of the skin due to curling hairs and shaving trauma.

It is an additional object of the present invention to provide treatment for shaved areas of the body while conditioning the skin during the day and overnight.

It is an additional object of the invention to prepare the skin for subsequent shaves, reducing the trauma involved with various shaving apparatuses.

Another object of the present invention is to treat and prevent skin irritation due primarily from shaving.

### DETAILED DESCRIPTION OF THE INVENTION

The composition of the present invention applied topically for the treatment and prevention of pseudofolliculitis barbae and irritation resulting from shaving with a manual razor or electric devices. It has been discovered that an efficient treatment of pseudofolliculitis barbae can be achieved with an acceptable cosmetic formulation (emulsion, solution, etc.) consisting of a combination of salicylic acid (or other classified BHA's), glycolic acid (or other classided AHA's), polyquaternium-10, and ingredients used for the purpose of anti-inflammatory or anti-irritant properties including but not limiting bisabolol, aloe, green tea, gorgonian extract, allantoin.

Alpha Hydroxy acids (AHA's) are a class of chemical compounds that consist of a carboxylic acid substituted with a hydroxy group on the adjacent carbon. Alpha-hydroxy acids are naturally occurring acids, derived from the sugars in particular plants. Some examples are Glycolic Acid (Sugar Cane), Lactic Acid (Milk), Tartaric Acid (Grapes), Citric Acid (Citrus Fruits), Malic Acid (Apples), and Mandelic Acid (Bitter Almonds). These acids work at the very base of the stratum corneum, dissolving the cement that holds dead skin cells together. This increases cell turnover and influences the structure of new stratum corneum being made. This results in skin that is more flexible, smoother, and more even in tone. At greater concentrations (12%-20%), deeper dermal effects, such as higher amounts of mucopolysaccharides and collagen and increased skin thickness, are observed. Eventually, alpha hydroxy acids will produce skin that is softer, smoother, less wrinkled, less dehydrated, and more even in skin tone. Alpha Hydroxy Acids may be present in the amounts of 0.1% to 15% of the total weight of the complete formulation.

Beta Hydroxy Acids (BHA's) namely but not limiting to salicylic acid, are also named from the molecular composition of the acid. These acids are unique in that they are readily absorbed by the fat properties of the skin. Skin is composed of water and fat molecules that limit penetration from the outside. Different from alpha hydroxy acid, this acid is unique in that it can penetrate into the pores of the skin. This helps clear the pores and exfoliate the skin. Beta hydroxy acid also has anti-inflammatory properties that result in less irritation, burning and stinging than alpha hydroxy acids. Its molecular structure, with the formula C₆H₄(OH)COOH, consists of a six-membered aromatic ring having a hydroxyl group (OH) and a carboxyl group (COOH) bonded to adjacent carbon atoms; as such, it is both a phenol and a carboxylic acid. Beta Hydroxy Acids may be present in the amounts of 0.1% to 2.0%. of the total weight of the complete formulation.

Polyquaternium-10 is a cationic, water-soluble substantive conditioner for hair care and skin care products; provides film formation and moisturization and controls the deposition of insoluble actives. Polyquaternium (or poly(diallyldimethylammonium chloride)) is a permanently charged polymer due to a quaternary amine. It is formed by reacting a trimethyl ammonium substituted epoxide onto the hydroxy groups of the hydroxyethyl cellulose backbone. The cationic charge on polyquaternium makes it substantive to keratinous hair and skin. In the present formulation, Polyquaternium-10 acts as a film former locking moisture into the skin to prevent dehydration of the skin and to soften the hair as it breaks through the skin reducing the chances of the hair growing back into the skin. Polyquaternium-10 may be present in the amounts of 0.01% to 5.0% of the total weight of the formulation.

Anti-inflammatory and anti-irritant ingredients refer to classes of compounds with properties of treatment that reduce inflammation (a localized protective reaction of tissue to irritation, injury, or infection, characterized by pain, redness, swelling, and sometimes loss of function) and soothe inflamed skin. These can be steroidal or non steroidal including but not limited to cortisone or botanicals such as hydrocortisone, gorgonian extract, bisabolol, glycyrrhizate, andoat beta glucan. Anti-inflammatory and anti-irritant ingredients can be used alone or in combination in the formula. The total amounts of anti-inflammatory and anti-irritant ingredients that may be present in the formulation are o.oo1% to 3%. Preferably the amounts range from 0.1 to 1.5%.

Silicones, or polysiloxanes, are inorganic-organic polymers with the chemical formula [R₂SiO]ₙ, where R=organic groups such as methyl, ethyl, and phenyl. These materials consist of an inorganic silicon-oxygen backbone ( . . . —Si—O—Si—O—Si—O— . . . ) with organic side groups attached to the silicon atoms, which are four-coordinate. In some cases organic side groups can be used to link two or more of these —Si—O— backbones together. By varying the —Si—O— chain lengths, side groups, and crosslinking, silicones can be synthesized with a wide variety of properties and compositions. This group includes ingredients such as alkylmethylsiloxanes, cyclopolysiloxanes (Dow Corning 200-100 CST Fluid, Dow Corning 200-50 CST Fluid, Dow Corning 245 Fluid, Dow Corning 246 Fluid, etc.). Total silicone percentage by weight in the complete formula is 1.0%-10.0%.

Hyperpigmentation is the result of an increased amount of melanin in the epidermis, the dermis, or both. This pigmentary change can be divided into two pathophysiologic processes: melanocytosis (increased number of melanocytes) and melanosis (increased amount of melanin). Depigmenting agents work best when melanosis or melanocytosis is restricted to the epidermis. Such ingredients used to treat hyperpigmentation including but not limited to hydroquinone, arbutin, bearberry extract, kojic acid, and glycyrrhizates. Other methods of depigmentation being used are chemical peels including but not limited to alpha hydroxy acids, beta hydroxy acids.

The formulation of the present invention is used as follows:

1. Clean the area to be shaved.
2. Shave using a manual razor or electric shaving devices.
3. At the completion of the shave, use an aftershave lotion/balm.
4. Apply the present invention to the shaved area and rub in until absorbed.

The invention should be applied daily to any affected area regardless of shaving. Furthermore, it should be applied at night to help condition the hair and to prepare the skin for subsequent shaves.

The product of the instant invention functions to treat or prevent pseudofolliculitis barbae, razor bumps, razor burns.

The following examples are illustrative of the formulation of the present invention and its method of use, but is not intended to limit its scope as claimed.

### EXAMPLE I

| | | | | |
|---|---|---|---|---|
| 1 | A | WATER | | 58.85 |
| 2 | A | DISODIUM EDTA | | 0.15 |
| 3 | A | ALLANTOIN | | 0.25 |
| 4 | A | POLYQUATERNIUM-10 | | 3.00 |
| 5 | A | HEXYLENE GLYCOL | | 1.50 |
| | | CAPRIC/CAPRYLIC | | |
| 6 | B | TRIGLYCERIDE | | 5.00 |
| | | CEATERYL ALCOHOL AND | | |
| 7 | B | CETEARETH-20 | | 2.50 |
| 8 | B | SALICYLIC ACID | | 2.00 |
| 9 | B | STEARETH-21 | | 2.00 |
| | | GLYCERYL STEARATE AND | | |
| 10 | B | PEG-100 STEARATE | | 3.00 |
| 11 | B | STEARETH-21 | | 0.20 |
| 12 | B | CYCLOPENTASILOXANE | | 5.00 |
| 13 | B | DIMETHICONE | | 2.00 |
| 14 | B | POLYMETHYLMETHACRYLATE | | 0.75 |
| 15 | C | PHENOXETHANOL | | 1.00 |
| 16 | C | FRAGRANCE | | 0.20 |
| 17 | C | ALOE VERA GEL | | 5.00 |
| 18 | D | POLYQUATERNIUM-31 | | 3.00 |
| 19 | E | GLYCOLIC ACID | | 2.50 |
| 20 | F | GORGONIAN EXTRACT | | 0.30 |
| 21 | F | BISABOLOL | | 0.20 |
| 22 | G | SODIUM HYDROXIDE | 50% | 1.60 |
| | | | SOLN | |
| | | TOTAL | | 100.00 |

### EXAMPLE II

| | | | | |
|---|---|---|---|---|
| 1 | A | WATER | | 58.20 |
| 2 | A | DISODIUM EDTA | | 0.15 |
| 3 | A | ALLANTOIN | | 0.50 |
| 4 | A | POLYQUATERNIUM-10 | | 2.00 |
| 5 | A | GLYCERIN | | 1.50 |
| 6 | B | ETHYLHEXYL PALMITATE | | 5.00 |
| | | CEATERYL ALCOHOL AND | | |
| 7 | B | CETEARETH-20 | | 2.50 |
| 8 | B | SALICYLIC ACID | | 1.50 |
| 9 | B | STEARETH-21 | | 2.00 |
| | | GLYCERYL STEARATE AND | | |
| 10 | B | PEG-100 STEARATE | | 3.00 |
| 11 | B | STEARETH-21 | | 0.20 |
| 12 | B | CYCLOMETHICONE | | 3.50 |
| 13 | B | DIMETHICONE | | 1.50 |
| 14 | B | POLYMETHYLMETHACRYLATE | | 0.75 |
| 15 | C | PHENOXETHANOL | | 1.00 |
| 16 | C | FRAGRANCE | | 0.20 |
| 17 | C | ALOE VERA GEL | | 5.00 |
| 18 | D | POLYQUATERNIUM-31 | | 3.00 |
| 19 | E | GLYCOLIC ACID | | 5.00 |
| 20 | F | GORGONIAN EXTRACT | | 0.50 |
| 21 | F | BISABOLOL | | 1.50 |
| 22 | G | SODIUM HYDROXIDE | 50% | 1.50 |
| | | | SOLN | |
| | | TOTAL | | 100.00 |

What is claimed is:

1. A formulation for reducing the occurrence of, treatment and prevention of pseudofolliculitis barbae when shaving with a manual razor or an electric device; said formulation consisting essentially of: an alpha hydroxy acid, beta hydroxy acid, dimethicone, volatile silicone, anti-inflammatories/anti-irritants, and polyquarternium-10.

2. A method of claim 1 wherein the said formulation consists of:
alpha hydroxy acids, fruit acids and derivatives thereof including but not limited to glycolic acid, lactic acid, glucuronic acid, galacturonic acid, gluconic acid, glucoheptonic acid, malic acid, muric acid, citric acid, saccharic acid, tartaric acid, tartronic acid, isocitric acid, dihydroxymaleic acid, dihydroxytartaric acid, and dihydroxyfumaric acid or derivatives thereof

3. A method of claim 1 wherein the said formulation contains a beta hydroxy acid or a derivative thereof including but not limited to salicylic acid

4. A method of claim 1 wherein the said formulation consists of high molecular weight silicones and volatile silicones.

5. A method of claim 4 wherein the silicone is a dimethicone that conforms to the Food and Drug Administration standards for an OTC skin protectant

6. A method of claim 4 wherein the silicone is a volatile silicone including but not limited to alkylmethylsiloxanes, cyclopolysiloxanes

7. A method of claim 1 for reducing the occurrence of and treatment of skin irritation resulting from shaving comprising of ingredients used specifically for its anti-inflammatory/anti-irritant activity or ingredients that have various functions with some anti-inflammatory/anti-irritant activity steroidal and non steroidal including but not limited to alpha-bisabolol, allantoin, aloe, glycyrrhizate, cortisone

8. A method of claim 6 wherein glycyrrhizate is ammonium, dipotassium, and/or calcium salts

9. A method of claim 1 wherein the said formulation consists of ingredients that promote even skin tone and depigmenting hyperpigmented areas on the skin including but not limited to alpha hydroxy acids, glycyrrhizate

10. A method of claim 1 wherein the said formulation consists of polyquaternium-10 to provide substantive conditioning to the skin

11. A method of claim 1 wherein the said formulation consists of a cationic or non-ionic emulsion, gel, creme, liquid, suspension or any other cosmetically acceptable vehicle.

12. A method of using said formulation for reducing the occurrence of, treatment and prevention of pseudofolliculitis barbae as part of a multi-step regimen consisting of shaving with a manual razor or electric device utilizing a pre-shave preparatory product, shaving gel/cream/foam and after shave balm.

13. A method of using said formulation for reducing the occurrence of, treatment and prevention of pseudofolliculitis barbae as a standalone treatment
